(19) United States
(12) Patent Application Publication
(10) Pub. No.: US 2020/0122127 A1
(43) Pub. Date: Apr. 23, 2020

(54) CATALYST FOR CHEMICAL LOOPING COMBUSTION

(71) Applicant: Research & Business Foundation Sungkyunkwan University, Suwon-si (KR)

(72) Inventors: Jong Wook BAE, Suwon-si (KR); Min Hye JEONG, Cheongju-si (KR)

(73) Assignee: Research & Business Foundation Sungkyunkwan University, Suwon-si (KR)

(21) Appl. No.: 16/656,653

(22) Filed: Oct. 18, 2019

(30) Foreign Application Priority Data
Oct. 19, 2018 (KR) 10-2018-0125079

(51) Int. Cl. B01J 23/83; C07C 5/41; B01J 37/03; B01J 37/08; B01J 23/00

(52) U.S. Cl. CPC B01J 23/83; C07C 5/412; C07C 11/04; B01J 37/08; B01J 23/002; B01J 37/031

(57) ABSTRACT

Disclosed are a catalyst for producing ethylene, and carbon monoxide from ethane and carbon dioxide via chemical looping process, a method for producing the same, and a chemical looping process using the same. The catalyst includes a complex metal oxide containing iron (Fe), cerium (Ce), and titanium (Ti). The catalyst has improved ethylene selectivity, carbon monoxide conversion, and stability.

### CROSS-REFERENCE TO RELATED APPLICATION

This application claims a benefit under 35 U.S.C. § 119(a) of Korean Patent Application No. 10-2018-0125079 filed on Oct. 19, 2018, in the Korean Intellectual Property Office, the entire disclosure of which is incorporated herein by reference for all purposes.

### BACKGROUND

### 1. Field

The present disclosure relates to a catalyst, and, more specifically, relates to a catalyst for producing ethylene from ethane, and carbon monoxide from carbon dioxide via chemical looping process, a method for producing the same, and a chemical looping process using the same.

### 2. Description of Related Art

Due to development of alternative energy resources due to limited reserves of fossil fuels and global warming due to carbon dioxide, development of renewable energy sources is an important issue around the world.

Methods for producing syngas using natural gas include partial oxidation of methane (POM), steam reforming of methane (SRM), and carbon dioxide reforming of methane (CDR).

An ethane cracking method is used as a method of synthesizing olefin as a main material of petrochemical industry. This method decomposes ethane contained in shale gas at high temperature of 1100° C. to obtain ethylene. In this method, serious problem of deactivation of catalysts due to coke deposition on catalyst surface may occur, which may increase process operation cost and investment cost.

In order to overcome this problem, the oxidative dehydrogenation (ODH) scheme has been proposed. However, because this method injects oxygen and ethane at the same time, a safety problem may occur and an air separation unit (ASU) may be necessary separately. In this method, a problem of a reduced selectivity of ethylene due to production of carbon dioxide as a by-product may occur. Therefore, there is a need for a new solution to solve this problem.

### SUMMARY

This Summary is provided to introduce a selection of concepts in a simplified form that are further described below in the Detailed Description. This Summary is not intended to identify all key features or essential features of the claimed subject matter, nor is it intended to be used alone as an aid in determining the scope of the claimed subject matter.

One purpose of the present disclosure is to provide a catalyst with improved ethylene selectivity, carbon monoxide conversion, and stability, a method for producing the same, and a chemical looping process using the same.

Purposes of the present disclosure are not limited to the above-mentioned purpose. Other purposes and advantages of the present disclosure as not mentioned above may be understood from following descriptions and more clearly understood from embodiments of the present disclosure. Further, it will be readily appreciated that the purposes and advantages of the present disclosure may be realized by features and combinations thereof as disclosed in the claims.

In a first aspect of the present disclosure, there is provided a catalyst for chemical looping process, wherein the chemical looping process produces ethylene, and carbon monoxide from ethane, and carbon dioxide, wherein the catalyst comprise a complex metal oxide containing iron (Fe), cerium (Ce), and titanium (Ti).

In one implementation of the first aspect, the complex metal oxide is composed such that a molar ratio of iron (Fe) to titanium (Ti) is in a range of 0.075 to 0.3, and a molar ratio of cerium (Ce) to titanium (Ti) is in a range of 0.075 to 0.3.

In one implementation of the first aspect, the complex metal oxide contains at least one of a rutile phase titania or an anatase phase titania.

In one implementation of the first aspect, the complex metal oxide contains ceria.

In one implementation of the first aspect, the complex metal oxide contains FeTi oxide containing iron, and titanium.

In one implementation of the first aspect, the complex metal oxide is composed of a carrier, and an active component substituted onto the carrier, wherein the carrier includes titania.

In one implementation of the first aspect, the active component includes a perovskite-structured material made of iron and cerium substituted onto titanium.

In one implementation of the first aspect, the catalyst has a conversion of ethane of 0.7% or greater, and has an ethylene selectivity of 62.0% or greater.

In a second aspect of the present disclosure, there is provided a method for producing a catalyst for chemical looping process, the method comprising: producing a first mixed solution by mixing iron precursor and cerium precursor with an aqueous solution containing titanium precursor; producing a second mixed solution by adding urea to the first mixed solution; filtering and drying the mixed metal oxide onto which the solution or suspension has been deposited; calcining the and producing a complex metal oxide by calcining the resulting mixture at a temperature suitable for the production of catalyst

In one implementation of the second aspect, the titanium precursor includes titanium oxysulfate (TiOSO₄).

In one implementation of the second aspect, the iron precursor includes iron chloride (FeCl₃).

In one implementation of the second aspect, the cerium precursor includes chloride cerium (CeCl₃).

In one implementation of the second aspect, the hot calcination is carried out at a temperature range of 500° C. to 1100° C.

In a third aspect of the present disclosure, there is provided a method for producing ethylene and carbon monoxide, the method comprising: disposing the catalyst for chemical looping process as defined above inside a chemical looping reactor; first heating the reactor to a reduction temperature or higher; injecting ethane-containing gas into the reactor to perform a reduction reaction; second heating the reactor; and injecting carbon dioxide-containing gas into the reactor to perform an oxidation reaction.

In one implementation of the third aspect, the first heating is carried out to a range of 450° C. to 650° C.

In one implementation of the third aspect, the method further comprises, after the injecting of the ethane-containing gas and before the second heating, converting an inside of the reactor into an inert gas atmosphere.

In one implementation of the third aspect, the second heating is carried out to a range of 600° C. to 800° C.

In one implementation of the third aspect, the disposing of the catalyst, the first heating, the injecting of the ethane-containing gas, the second heating and the injecting of the carbon dioxide-containing gas are sequentially repeated.

Effects of the present disclosure are as follows but are not limited thereto.

The catalyst in accordance with the present disclosure includes a composite containing iron, cerium, and titanium. The catalyst in accordance with the present disclosure may be used in a chemical looping process where oxidation and reduction are performed sequentially. In particular, the catalyst in accordance with the present disclosure may be used for the production of ethylene and carbon monoxide from ethane and carbon dioxide respectively via the chemical looping process. The catalyst in accordance with the present disclosure may have reaction stability, catalyst activity, and product conversion superior to those of the conventional catalysts.

### BRIEF DESCRIPTION OF THE DRAWINGS

The accompanying drawings, which are incorporated in and form a part of this specification and in which like numerals depict like elements, illustrate embodiments of the present disclosure and, together with the description, serve to explain the principles of the disclosure.

FIG. 1 shows a result of chemical looping process according to one embodiment of the present disclosure.

FIG. 2 shows a result of catalyst analysis according to one embodiment of the present disclosure.

FIG. 3 shows a result of catalyst analysis according to one embodiment of the present disclosure.

### DETAILED DESCRIPTIONS

Examples of various embodiments are illustrated and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims. Further, descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure.

The terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises”, “comprising”, “includes”, and “including” when used in this specification, specify the presence of the stated features, integers, operations, elements, and/or components, but do not preclude the presence or addition of one or more other features, integers, operations, elements, components, and/or portions thereof. Expression such as “at least one of” when preceding a list of elements may modify the entire list of elements and may not modify the individual elements of the list.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The catalyst for chemical looping process in accordance with the present disclosure includes a complex metal oxide including iron (Fe), cerium (Ce), and titanium (Ti). On the catalyst in accordance with the present disclosure, selective conversion reaction of ethane among paraffin based hydrocarbons into ethylene via dehydrogenation reaction may be carried out, and reaction in which carbon monoxide is produced using carbon dioxide as an oxidant may occur. In other words, the catalyst in accordance with the present disclosure may be used to produce ethylene, and carbon monoxide from ethane and carbon dioxide via chemical looping process. The catalyst in accordance with the present disclosure may have reaction stability, catalyst activity, and product conversion superior to those of the conventional catalysts.

In one embodiment, the complex metal oxide may be composed such that a molar ratio of iron to titanium may be in a range of 0.075 to 0.3, and a molar ratio of cerium to titanium may be in a range of 0.075 to 0.3. For example, the complex metal oxide may be composed such that molar ratios of iron and cerium to titanium may be 0.075, and 0.075 respectively, 0.150, and 0.075 respectively, 0.225, and 0.075 respectively, 0.3, and 0.075 respectively, 0.075, and 0.15 respectively, or 0.075, and 0.3 respectively. For example, when each of the molar ratios of iron and cerium to titanium is smaller than 0.075, the number of the oxygen defects and thus the active points in the catalyst may be small, resulting in low reaction activity. When each of the molar ratios of iron and cerium to titanium is greater than 0.3, coke and catalyst agglomerating may cause deterioration of selectivity to ethylene and stability.

In one embodiment, at least one of iron, cerium, and titanium included in the complex metal oxide may be present in an oxide form thereof. For example, the complex metal oxide may include at least one of iron oxide, cerium oxide (or ceria, CeO₂), and titanium oxide (or titania, TiO₂). Transition metal oxides such as cerium oxide, and iron oxide have the property of easy electron exchange and, at the same time, are inexpensive and well maintained at high temperatures. In particular, ceria may be used for chemical looping process due to its high oxygen carrying capacity (OSC) as oxygens are released from lattices to form vacancies of negative ions. As a result, the chemical looping process maybe performed due to the ceria without a separate oxygen supply.

Catalyst in accordance with the present disclosure may be present in a mixture form of various phases obtained by controlling the molar ratios of iron, cerium, and titanium, and a calcination temperature during production. For example, the catalyst in accordance with the present disclosure may be present may in mixed powders including the various phases.

In one embodiment, the complex metal oxide may include titania having at least one phase of a rutile phase and an anatase phase. For example, the complex metal oxide may include titania in an anatase phase or titania in a rutile phase. Alternatively, the titania in the anatase phase and titania in the rutile phase may be mixed with each other.

In one embodiment, the complex metal oxide may include FeTi oxide including iron and titanium. For example, the complex metal oxide may include FeTi oxide having one or more phases of Fe₂TiO₅, Fe_{1.696}O₃Ti_{0.228}, and pseudobrookite phase.

In one embodiment, the catalyst may include the complex metal oxide composed of a carrier and an active component supported on the carrier.

In one embodiment, the carrier of the complex metal oxide includes titania. The active component may include cerium and iron. For example, the complex metal oxide may include a solid of Perovskite in which titanium are substituted with iron and cerium. Alternatively, a mixture of cerium oxide and iron oxide may be present as an active component on the carrier including titania.

Transition metal oxides are used as redox catalysts due to their easy electron exchange, low cost, and high thermal retention. A FeCeTi composite oxide catalyst whose active ingredients, specifically, active metals include iron (Fe), cerium (Ce), and titanium (Ti) may be short-range order species having mutual interactions between metals and thus may act as an active component or active point. In particular, an oxygen in a lattice of CeO₂ is separated therefrom to form a negative ion vacancy to achieve a high oxygen carrying capacity (OSC). Thus, the FeCeTi composite oxide catalyst has been widely used in the chemical looping process. When iron ions with smaller ionic radii and lower valence state than cerium ions replace lattices of CeO₂, oxygen defects may be generated and strong structural distortions and smaller oxygen defect generation energy may be generated. Thus, the oxygen vacancy may act as an active point. A solid solution of a perovskite structure of iron and titanium may allow change in electronic properties of each metal to increase the reactivity, and stability.

In one embodiment, the catalyst may include oxygen donor particles. For example, the complex metal oxide may be the oxygen donor particle. The catalyst for chemical looping process in accordance with the present disclosure may include the complex metal oxide with the oxygen donor particles in place of a separate direct oxygen injection process, thereby to increase production efficiency of ethylene from ethane and, at the same time, to reduce carbon dioxide emission.

In one embodiment, the catalyst has an ethane conversion of greater than or equal to 0.7%, and at the same time, has an ethylene selectivity of 62.0% or greater.

In one embodiment, the complex metal oxide has a specific surface area of 0.02 m²/g to 50.6 m²/g has a pore size in a range of 6 nm to 39.8 nm. For example, the specific surface area may be in a range of 20 m²/g to 50 m²/g. The pore size may be in a range of 8.5 nm to 18 nm.

A method for producing a catalyst for chemical looping process in accordance with the present disclosure includes a first step of producing a first mixed solution by mixing iron precursor, and cerium precursor in an aqueous solution containing titanium precursor; a second step of producing a second mixed solution by adding urea to the first mixed solution; a third step of filtering and drying the mixed metal oxide onto which the solution or suspension has been deposited; and a fourth step of calcining the resulting mixture at a temperature suitable for the production of catalyst.

In one embodiment, the titanium precursor may include titanium oxysulfate (TiOSO₄) but may not be limited thereto. In one embodiment, the iron precursor may be iron chloride (FeCl₃), but is not limited thereto. In one embodiment, the cerium precursor may include cerium chloride (CeCl₃), but is not limited thereto. For example, the first step may include producing the first mixed solution by mixing iron chloride, and cerium chloride in an aqueous solution containing titanium oxysulfate and stirring the mixture at room temperature.

In one embodiment, the first mixed solution may be composed such that each of molar ratios of iron and cerium to titanium may be in a range of 0.075 to 0.3.

In one embodiment, the second step of producing the second mixed solution by adding urea to the first mixed solution may include producing the second mixed solution by heating the first mixed solution and then adding urea thereto for synthesis. In one embodiment, the synthesis may be performed for 5 hours to 9 hours. For example, the synthesis may be performed for 7 hours.

In one embodiment, the third step may cool and filter the second mixed solution and then collect the precipitate to produce the first precipitate. In one embodiment, after the third step, and before the fourth step, the method may further include a step of drying the first precipitate.

In one embodiment, the fourth step may produce the complex metal oxide by hot calcination of the dried first precipitate.

In one embodiment, the hot calcination of the fourth step may be performed at a temperature range of 500° C. to 1100° C. For example, the hot calcination may be performed at 600° C., 700° C., 800° C., 900° C., and 1000° C. In one embodiment, adjusting the calcination temperature to produce the catalyst may improve ethylene selectivity, and carbon monoxide conversion in the chemical looping process using the catalyst.

The chemical looping process in accordance with the present disclosure may include (a) placing the catalyst for the chemical looping process in accordance with the present disclosure inside a chemical looping reactor; (b) first-heating the reactor to a temperature above a reducing temperature; (c) injecting ethane-containing gas into the reactor to perform a reduction reaction; (d) second-heating the reactor; (e) injecting carbon dioxide-containing gas into the reactor to perform an oxidation reaction, thereby to produce ethylene, and carbon monoxide.

In one embodiment, the chemical looping reactor may include a fixed bed reactor but is not limited thereto. In one embodiment, the temperature inside the reactor may be measured by placing a thermocouple in the reactor to be positioned above the catalyst. In one embodiment, the catalyst may be placed in a center of the reactor. In one embodiment, the reactor may be heated using an electric furnace. For example, a PID controller may be used to externally control the temperature of the reactor.

In one embodiment, the first heating of the step (b) may be carried out at a temperature of 450° C. to 650° C. For example, the first heating may raise the reactor to a reduction temperature. For example, the reactor may be heated to 550° C. via the first heating. In one embodiment, the step (b) may be performed in a nitrogen gas atmosphere.

In one embodiment, the step (c) may be performed after an isothermal condition in which the reducing temperature is maintained is met. In one embodiment, the ethane-containing gas injected into the reactor may include ethane, and nitrogen. For example, 20 mol % of ethane may be included therein. In one embodiment, the reduction of the step (c) may be carried out for 3 to 5 hours. For example, the reduction may be performed for 4 hours.

In one embodiment, after the step (c) and, before the step (d), the method may further include a step of converting an inside atmosphere of the reactor to an inert gas atmosphere. For example, after the step (c) and before the step (d), the method may further include blocking the ethane-containing gas to be injected into the reactor and excluding the ethane and injecting only nitrogen gas thereto when the reduction reaction is finished, thereby to remove residual gas in the reactor.

In one embodiment, the step (d) of the second heating of the reactor may be performed after removing the residual gas inside the reactor. For example, the second heating may include heating of the reactor to a range of 600° C. to 800° C.

In one embodiment, after the step (d), the step (e) may be performed to inject the carbon dioxide-containing gas into the reactor to perform an oxidation reaction. In one embodiment, the carbon dioxide-containing gas in the step (e) may include a mixture of carbon dioxide, and nitrogen gas. For example, carbon dioxide may be included therein at a content of 20 mol %. For example, the step (e) may perform the oxidation reaction in a carbon dioxide and nitrogen mixed gas atmosphere.

In one embodiment, the chemical looping process may include repeating the steps (b) to (e) sequentially.

According to one embodiment of the present disclosure, the catalyst in accordance with the present disclosure was produced as follows.

### PRESENT EXAMPLE 1

### Production of Catalyst 1

(iron/cerium/titanium molar ratio=0.075/0.075/1, calcination temperature 600° C.)

First, 1.2 mmol of titanium oxysulfate (TiOSO₄) was dissolved in 1500 ml of distilled water in a 2 L three-necked flask. Then, 0.09 mmol of iron chloride (FeCl₃), and cerium chloride (CeCl₃) were added to the solution which in turn was stirred at room temperature for 1 hour. After heating a temperature of a heating mantle to 90° C., urea was added to the solution to perform synthesis for 7 hours. After the synthesis was completed and then the temperature was lowered to room temperature, the mixture solution was filtered using a decompression filter. The precipitate was then collected and placed in a vacuum oven maintained at 80° C. and dried for 24 hours. The dried precipitate was collected and heated at a rate of 1° C./min from room temperature to 600° C., and then was kept at 600° C. for 6 hours, thereby to yield a catalyst 1 in accordance with the present disclosure.

In this connection, a carrier of the complex metal oxide includes titania. An active component on the carrier may include a perovskite structured solid in which iron, and cerium are substituted for titanium. Alternatively, the cerium and iron oxides as the active component may be present in the mixed form on the carrier including the titania.

The catalyst 1 was composed such that the iron/cerium/titanium molar ratio is 0.075/0.075/1. The specific surface area of the catalyst 1 was 48.5 m²/g. The average pore size thereof was found to be 9.4 nm. X-ray diffraction (XRD) analysis showed that the catalyst 1 includes mixed powders of the anatase phase titania and Fe_{1.696}O₃Ti_{0.228} phase titania.

Subsequently, chemical looping process according to one embodiment using the catalyst 1 was performed. In order to perform the reaction, an Inconel fixed bed reactor having an inner diameter of 7 mm, an outer diameter of 9.5 mm, and a height of 420 mm was used. A temperature inside the reactor was measured after placing a 1/16 inch thermocouple from a top of the reactor into the reactor and positioning the thermocouple above the catalyst bed. A 207 mm long 4/16 tube was disposed from a bottom of the reactor in order to place the catalyst in the center of the reactor. Then, 1 g of catalyst, and 0.15 g of glass wool were charged into the reactor through the tube. Then, the reaction according to one embodiment was carried out. In this connection. The reactor was supplied with heat using an electrical furnace (World Energy company). The thermocouple external to the furnace was used to measure the temperature. Then, the temperature of the reactor may be externally controlled by a PID controller based on the measured temperature.

Only nitrogen was injected to the reactor while increasing the reactor temperature from room temperature to a reduction temperature of 550° C. Then, an isothermal condition is established in which the reduction temperature in accordance with the present disclosure is maintained at 550° C. Then, a total gas flow rate into the reactor was changed to a 30 cc/min to adjust a content of the ethane gas to 20 mol % (ethane 6 cc/min, nitrogen 24 cc/min), and then, a reduction reaction was performed for 4 hours. After the reduction reaction was completed, the flow of ethane was shut off and only nitrogen was flowed into the reactor to remove residual gas inside the reactor.

Thereafter, the temperature inside the reactor was set to 700° C. Then, the oxidation reaction was performed under a nitrogen gas atmosphere having the same gas flow rate as in the reduction reaction and containing 20 mol % of carbon dioxide. Termination of the oxidation reaction was calculated based on a timing at which generation of carbon monoxide as a product was not identified by GC (gas chromatography) as an analyzer. The produced gas was analyzed by gas chromatography connected to the reactor. A thermal conductivity detector (TCD) and flame ionization detector (FID) were used.

Results of catalyst activity using the catalyst 1 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 2

### Production of Catalyst 2

(iron/cerium/titanium molar ratio=0.075/0.075/1, calcination temperature 700° C.)

A catalyst 2 was produced in the same manner as the Present Example 1 excerpt that the hot calcination condition was adjusted to 700° C. and the precipitate was maintained for 6 hours. The catalyst 2 was composed such that the iron/cerium/titanium molar ratio is 0.075/0.075/1. The specific surface area of the catalyst 2 was 23.6 m²/g. The average pore size thereof was found to be 12.4 nm. X-ray diffraction (XRD) analysis showed that the catalyst 2 includes mixed powders of a rutile phase titania and a pseudobrookite (Fe₂TiO₅) phase titania and a Fe_{1.696}O₃Ti_{0.228} phase titania.

Subsequently, chemical looping process according to one embodiment using the catalyst 2 was performed. Results of catalyst activity using the catalyst 2 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 3

### Production of Catalyst 3

(iron/cerium/titanium molar ratio=0.075/0.075/1, calcination temperature 800° C.)

A catalyst 3 was produced in the same manner as the Present Example 1 excerpt that the hot calcination condition was adjusted to 800° C. and the precipitate was maintained for 6 hours. The catalyst 3 was composed such that the iron/cerium/titanium molar ratio is 0.075/0.075/1. The specific surface area of the catalyst 3 was 0.69 m²/g. The average pore size thereof was found to be 13.5 nm. X-ray diffraction (XRD) analysis showed that the catalyst 3 includes mixed powders of a rutile phase titania and a pseudobrookite (Fe₂TiO₅) phase titania.

Subsequently, chemical looping process according to one embodiment using the catalyst 3 was performed. Results of catalyst activity using the catalyst 3 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 4

### Production of Catalyst 4

(iron/cerium/titanium molar ratio=0.075/0.075/1, calcination temperature 900° C.)

A catalyst 4 was produced in the same manner as the Present Example 1 excerpt that the hot calcination condition was adjusted to 900° C. and the precipitate was maintained for 6 hours. The catalyst 4 was composed such that the iron/cerium/titanium molar ratio is 0.075/0.075/1. The specific surface area of the catalyst 4 was 0.23 m²/g. The average pore size thereof was found to be 37.9 nm. X-ray diffraction (XRD) analysis showed that the catalyst 4 includes mixed powders of a rutile phase titania and a pseudobrookite (Fe₂TiO₅) phase titania.

Subsequently, chemical looping process according to one embodiment using the catalyst 4 was performed. Results of catalyst activity using the catalyst 4 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 5

### Production of Catalyst 5

(iron/cerium/titanium molar ratio=0.075/0.075/1, calcination temperature 1000° C.)

A catalyst 5 was produced in the same manner as the Present Example 1 excerpt that the hot calcination condition was adjusted to 1000° C. and the precipitate was maintained for 6 hours. The catalyst 5 was composed such that the iron/cerium/titanium molar ratio is 0.075/0.075/1. The specific surface area of the catalyst 5 was 0.07 m²/g. The average pore size thereof was found to be 8.5 nm. X-ray diffraction (XRD) analysis showed that the catalyst 5 includes mixed powders of a rutile phase titania and a pseudobrookite (Fe₂TiO₅) phase titania.

Subsequently, chemical looping process according to one embodiment using the catalyst 5 was performed. Results of catalyst activity using the catalyst 5 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 6

### Production of Catalyst 6

(iron/cerium/titanium molar ratio=0.150/0.075/1, calcination temperature 600° C.)

A catalyst 6 was produced in the same manner as the Present Example 1 excerpt that 0.18 mmol of iron chloride was used and the hot calcination condition was adjusted to 600° C. and the precipitate was maintained for 6 hours. The catalyst 6 was composed such that the iron/cerium/titanium molar ratio is 0.150/0.075/1. The specific surface area of the catalyst 6 was 34.9 m²/g. The average pore size thereof was found to be 9.1 nm. X-ray diffraction (XRD) analysis showed that the catalyst 7 includes powders of an anatase phase titania.

Subsequently, chemical looping process n according to one embodiment using the catalyst 6 was performed. Results of catalyst activity using the catalyst 6 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 7

### Production of Catalyst 7

(iron/cerium/titanium molar ratio=0.150/0.075/1, calcination temperature 700° C.)

A catalyst 7 was produced in the same manner as the Present Example 1 excerpt that 0.18 mmol of iron chloride was used and the hot calcination condition was adjusted to 700° C. and the precipitate was maintained for 6 hours. The catalyst 7 was composed such that the iron/cerium/titanium molar ratio is 0.150/0.075/1. The specific surface area of the catalyst 7 was 7.5 m²/g. The average pore size thereof was found to be 11.1 nm. X-ray diffraction (XRD) analysis showed that the catalyst 7 includes mixed powders of a pseudobrookite (Fe₂TiO₅) phase titania and an anatase phase titania.

Subsequently, chemical looping process according to one embodiment using the catalyst 7 was performed. Results of catalyst activity using the catalyst 7 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 8

### Production of Catalyst 8

(iron/cerium/titanium molar ratio=0.150/0.075/1, calcination temperature 800° C.)

A catalyst 8 was produced in the same manner as the Present Example 1 excerpt that 0.18 mmol of iron chloride was used and the hot calcination condition was adjusted to 800° C. and the precipitate was maintained for 6 hours. The catalyst 8 was composed such that the iron/cerium/titanium molar ratio is 0.150/0.075/1. The specific surface area of the catalyst 8 was 0.17 m²/g. The average pore size thereof was found to be 39.8 nm. X-ray diffraction (XRD) analysis showed that the catalyst 8 includes mixed powders of a pseudobrookite (Fe₂TiO₅) phase titania and an anatase phase titania and a rutile phase titania.

Subsequently, chemical looping process according to one embodiment using the catalyst 8 was performed. Results of catalyst activity using the catalyst 8 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 9

### Production of Catalyst 9

(iron/cerium/titanium molar ratio=0.150/0.075/1, calcination temperature 900° C.)

A catalyst 9 was produced in the same manner as the Present Example 1 excerpt that 0.18 mmol of iron chloride was used and the hot calcination condition was adjusted to 900° C. and the precipitate was maintained for 6 hours. The catalyst 9 was composed such that the iron/cerium/titanium molar ratio is 0.150/0.075/1. The specific surface area of the catalyst 9 was 0.03 m²/g. The average pore size thereof was found to be 10.4 nm. X-ray diffraction (XRD) analysis showed that the catalyst 9 includes mixed powders of a pseudobrookite (Fe₂TiO₅) phase titania and a rutile phase titania.

Subsequently, chemical looping process according to one embodiment using the catalyst 9 was performed. Results of catalyst activity using the catalyst 9 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 10

### Production of Catalyst 10

(iron/cerium/titanium molar ratio=0.150/0.075/1, calcination temperature 1000° C.)

A catalyst 10 was produced in the same manner as the Present Example 1 excerpt that 0.18 mmol of iron chloride was used and the hot calcination condition was adjusted to 1000° C. and the precipitate was maintained for 6 hours. The catalyst 10 was composed such that the iron/cerium/titanium molar ratio is 0.150/0.075/1. The specific surface area of the catalyst 10 was 0.02 m²/g. X-ray diffraction (XRD) analysis showed that the catalyst 10 includes mixed powders of a pseudobrookite (Fe₂TiO₅) phase titania and a rutile phase titania.

Subsequently, chemical looping process according to one embodiment using the catalyst 10 was performed. Results of catalyst activity using the catalyst 10 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 11

### Production of Catalyst 11

(iron/cerium/titanium molar ratio=0.225/0.075/1, calcination temperature 600° C.)

A catalyst 11 was produced in the same manner as the Present Example 1 excerpt that 0.27 mmol of iron chloride was used and the hot calcination condition was adjusted to 600° C. and the precipitate was maintained for 6 hours. The catalyst 11 was composed such that the iron/cerium/titanium molar ratio is 0.225/0.075/1. The specific surface area of the catalyst 11 was 44.3 m²/g. The average pore size thereof was found to be 16.3 nm. X-ray diffraction (XRD) analysis showed that the catalyst 11 includes mixed powders of an anatase phase titania, and a Fe_{1.696}O₃Ti_{0.228} phase titania.

Subsequently, chemical looping process according to one embodiment using the catalyst 11 was performed. Results of catalyst activity using the catalyst 11 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 12

### Production of Catalyst 12

(iron/cerium/titanium molar ratio=0.225/0.075/1, calcination temperature 700° C.)

A catalyst 12 was produced in the same manner as the Present Example 1 excerpt that 0.27 mmol of iron chloride was used and the hot calcination condition was adjusted to 700° C. and the precipitate was maintained for 6 hours. The catalyst 12 was composed such that the iron/cerium/titanium molar ratio is 0.225/0.075/1. The specific surface area of the catalyst 12 was 17.5 m²/g. The average pore size thereof was found to be 17.9 nm. X-ray diffraction (XRD) analysis showed that the catalyst 12 includes mixed powders of an anatase phase titania, and a Fe_{1.696}O₃Ti_{0.228} phase titania and a rutile phase titania.

Subsequently, chemical looping process according to one embodiment using the catalyst 12 was performed. Results of catalyst activity using the catalyst 12 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 13

### Production of Catalyst 13

(iron/cerium/titanium molar ratio=0.225/0.075/1, calcination temperature 800° C.)

A catalyst 13 was produced in the same manner as the Present Example 1 excerpt that 0.27 mmol of iron chloride was used and the hot calcination condition was adjusted to 800° C. and the precipitate was maintained for 6 hours. The catalyst 13 was composed such that the iron/cerium/titanium molar ratio is 0.225/0.075/1. The specific surface area of the catalyst 13 was 6.03 m²/g. The average pore size thereof was found to be 12.7 nm. X-ray diffraction (XRD) analysis showed that the catalyst 13 includes mixed powders of a rutile phase titania and a pseudobrookite (Fe₂TiO₅) phase titania.

Subsequently, chemical looping process according to one embodiment using the catalyst 13 was performed. Results of catalyst activity using the catalyst 13 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 14

### Production of Catalyst 14

(iron/cerium/titanium molar ratio=0.225/0.075/1, calcination temperature 900° C.)

A catalyst 14 was produced in the same manner as the Present Example 1 excerpt that 0.27 mmol of iron chloride was used and the hot calcination condition was adjusted to 900° C. and the precipitate was maintained for 6 hours. The catalyst 14 was composed such that the iron/cerium/titanium molar ratio is 0.225/0.075/1. The specific surface area of the catalyst 14 was 3.26 m²/g. The average pore size thereof was found to be 23.8 nm. X-ray diffraction (XRD) analysis showed that the catalyst 14 includes mixed powders of a rutile phase titania and a pseudobrookite (Fe₂TiO₅) phase titania.

Subsequently, chemical looping process according to one embodiment using the catalyst 14 was performed. Results of catalyst activity using the catalyst 14 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 15

### Production of Catalyst 15

(iron/cerium/titanium molar ratio=0.225/0.075/1, calcination temperature 1000° C.)

A catalyst 15 was produced in the same manner as the Present Example 1 excerpt that 0.27 mmol of iron chloride was used and the hot calcination condition was adjusted to 1000° C. and the precipitate was maintained for 6 hours. The catalyst 15 was composed such that the iron/cerium/titanium molar ratio is 0.225/0.075/1. The specific surface area of the catalyst 15 was 1.18 m²/g. The average pore size thereof was found to be 33.0 nm. X-ray diffraction (XRD) analysis showed that the catalyst 15 includes mixed powders of a rutile phase titania and a pseudobrookite (Fe₂TiO₅) phase titania.

Subsequently, chemical looping process according to one embodiment using the catalyst 15 was performed. Results of catalyst activity using the catalyst 15 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 16

### Production of Catalyst 16

(iron/cerium/titanium molar ratio=0.300/0.075/1, calcination temperature 600° C.)

A catalyst 16 was produced in the same manner as the Present Example 1 excerpt that 0.36 mmol of iron chloride was used and the hot calcination condition was adjusted to 600° C. and the precipitate was maintained for 6 hours. The catalyst 16 was composed such that the iron/cerium/titanium molar ratio is 0.300/0.075/1. The specific surface area of the catalyst 16 was 50.6 m²/g. The average pore size thereof was found to be 13.1 nm. X-ray diffraction (XRD) analysis showed that the catalyst 16 includes mixed powders of an anatase phase titania and a pseudobrookite (Fe₂TiO₅) phase titania.

Subsequently, chemical looping process according to one embodiment using the catalyst 16 was performed. Results of catalyst activity using the catalyst 16 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 17

### Production of Catalyst 17

(iron/cerium/titanium molar ratio=0.300/0.075/1, calcination temperature 700° C.)

A catalyst 17 was produced in the same manner as the Present Example 1 excerpt that 0.36 mmol of iron chloride was used and the hot calcination condition was adjusted to 700° C. and the precipitate was maintained for 6 hours. The catalyst 17 was composed such that the iron/cerium/titanium molar ratio is 0.300/0.075/1. The specific surface area of the catalyst 17 was 18.3 m²/g. The average pore size thereof was found to be 20.2 nm. X-ray diffraction (XRD) analysis showed that the catalyst 17 includes mixed powders of a pseudobrookite (Fe₂TiO₅) phase titania and an anatase phase titania and a rutile phase titania.

Subsequently, chemical looping process according to one embodiment using the catalyst 17 was performed. Results of catalyst activity using the catalyst 17 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 18

### Production of Catalyst 18

(iron/cerium/titanium molar ratio=0.300/0.075/1, calcination temperature 800° C.)

A catalyst 18 was produced in the same manner as the Present Example 1 excerpt that 0.36 mmol of iron chloride was used and the hot calcination condition was adjusted to 800° C. and the precipitate was maintained for 6 hours. The catalyst 18 was composed such that the iron/cerium/titanium molar ratio is 0.300/0.075/1. The specific surface area of the catalyst 18 was 3.81 m²/g. The average pore size thereof was found to be 20.4 nm. X-ray diffraction (XRD) analysis showed that the catalyst 18 includes mixed powders of a pseudobrookite (Fe₂TiO₅) phase titania and a rutile phase titania.

Subsequently, chemical looping process according to one embodiment using the catalyst 18 was performed. Results of catalyst activity using the catalyst 18 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 19

### Production of Catalyst 19

(iron/cerium/titanium molar ratio=0.300/0.075/1, calcination temperature 900° C.)

A catalyst 19 was produced in the same manner as the Present Example 1 excerpt that 0.36 mmol of iron chloride was used and the hot calcination condition was adjusted to 900° C. and the precipitate was maintained for 6 hours. The catalyst 19 was composed such that the iron/cerium/titanium molar ratio is 0.300/0.075/1. The specific surface area of the catalyst 19 was 2.25 m²/g. The average pore size thereof was found to be 8.6 nm. X-ray diffraction (XRD) analysis showed that the catalyst 19 includes mixed powders of a pseudobrookite (Fe₂TiO₅) phase titania and a rutile phase titania, and ceria (CeO₂).

Subsequently, chemical looping process according to one embodiment using the catalyst 19 was performed. Results of catalyst activity using the catalyst 19 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 20

### Production of Catalyst 20

(iron/cerium/titanium molar ratio=0.300/0.075/1, calcination temperature 1000° C.)

A catalyst 20 was produced in the same manner as the Present Example 1 excerpt that 0.36 mmol of iron chloride was used and the hot calcination condition was adjusted to 1000° C. and the precipitate was maintained for 6 hours. The catalyst 20 was composed such that the iron/cerium/titanium molar ratio is 0.300/0.075/1. The specific surface area of the catalyst 20 was 0.82 m²/g. The average pore size thereof was found to be 8.6 nm. X-ray diffraction (XRD) analysis showed that the catalyst 20 includes mixed powders of a pseudobrookite (Fe₂TiO₅) phase titania and a rutile phase titania and ceria.

Subsequently, chemical looping process according to one embodiment using the catalyst 20 was performed. Results of catalyst activity using the catalyst 20 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 21

### Production of Catalyst 21

(iron/cerium/titanium molar ratio=0.075/0.150/1, calcination temperature 600° C.)

A catalyst 21 was produced in the same manner as the Present Example 1 excerpt that 0.18 mmol of cerium chloride was used and the hot calcination condition was adjusted to 600° C. and the precipitate was maintained for 6 hours. The catalyst 21 was composed such that the iron/cerium/titanium molar ratio is 0.075/0.150/1. The specific surface area of the catalyst 21 was 31.0 m²/g. The average pore size thereof was found to be 12.5 nm. X-ray diffraction (XRD) analysis showed that the catalyst 21 includes mixed powders of an anatase phase titania and ceria.

Subsequently, chemical looping process according to one embodiment using the catalyst 21 was performed. Results of catalyst activity using the catalyst 21 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 22

### Production of Catalyst 22

(iron/cerium/titanium molar ratio=0.075/0.150/1, calcination temperature 700° C.)

A catalyst 22 was produced in the same manner as the Present Example 1 excerpt that 0.18 mmol of cerium chloride was used and the hot calcination condition was adjusted to 700° C. and the precipitate was maintained for 6 hours. The catalyst 22 was composed such that the iron/cerium/titanium molar ratio is 0.075/0.150/1. The specific surface area of the catalyst 22 was 19.9 m²/g. The average pore size thereof was found to be 17.7 nm. X-ray diffraction (XRD) analysis showed that the catalyst 22 includes mixed powders of an anatase phase titania and ceria.

Subsequently, chemical looping process according to one embodiment using the catalyst 22 was performed. Results of catalyst activity using the catalyst 22 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 23

### Production of Catalyst 23

(iron/cerium/titanium molar ratio=0.075/0.150/1, calcination temperature 800° C.)

A catalyst 23 was produced in the same manner as the Present Example 1 excerpt that 0.18 mmol of cerium chloride was used and the hot calcination condition was adjusted to 800° C. and the precipitate was maintained for 6 hours. The catalyst 23 was composed such that the iron/cerium/titanium molar ratio is 0.075/0.150/1. The specific surface area of the catalyst 23 was 7.2 m²/g. The average pore size thereof was found to be 22.3 nm. X-ray diffraction (XRD) analysis showed that the catalyst 23 includes mixed powders of a rutile phase titania and a pseudobrookite (Fe₂TiO₅) phase titania and ceria.

Subsequently, chemical looping process according to one embodiment using the catalyst 23 was performed. Results of catalyst activity using the catalyst 23 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 24

### Production of Catalyst 24

(iron/cerium/titanium molar ratio=0.075/0.150/1, calcination temperature 900° C.)

A catalyst 24 was produced in the same manner as the Present Example 1 excerpt that 0.18 mmol of cerium chloride was used and the hot calcination condition was adjusted to 900° C. and the precipitate was maintained for 6 hours. The catalyst 24 was composed such that the iron/cerium/titanium molar ratio is 0.075/0.150/1. The specific surface area of the catalyst 24 was 2.8 m²/g. The average pore size thereof was found to be 34.9 nm. X-ray diffraction (XRD) analysis showed that the catalyst 24 includes mixed powders of a rutile phase titania and a pseudobrookite (Fe₂TiO₅) phase titania and ceria.

Subsequently, chemical looping process according to one embodiment using the catalyst 24 was performed. Results of catalyst activity using the catalyst 24 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 25

### Production of Catalyst 25

(iron/cerium/titanium molar ratio=0.075/0.150/1, calcination temperature 1000° C.)

A catalyst 25 was produced in the same manner as the Present Example 1 excerpt that 0.18 mmol of cerium chloride was used and the hot calcination condition was adjusted to 1000° C. and the precipitate was maintained for 6 hours. The catalyst 25 was composed such that the iron/cerium/titanium molar ratio is 0.075/0.150/1. The specific surface area of the catalyst 25 was 1.0 m²/g. The average pore size thereof was found to be 33.3 nm. X-ray diffraction (XRD) analysis showed that the catalyst 25 includes mixed powders of a rutile phase titania and a pseudobrookite (Fe₂TiO₅) phase titania and ceria.

Subsequently, chemical looping process according to one embodiment using the catalyst 25 was performed. Results of catalyst activity using the catalyst 25 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 26

### Production of Catalyst 26

(iron/cerium/titanium molar ratio=0.075/0.300/1, calcination temperature 600° C.)

A catalyst 26 was produced in the same manner as the Present Example 1 excerpt that 0.36 mmol of cerium chloride was used and the hot calcination condition was adjusted to 600° C. and the precipitate was maintained for 6 hours. The catalyst 26 was composed such that the iron/cerium/titanium molar ratio is 0.075/0.300/1. The specific surface area of the catalyst 26 was 9.3 m²/g. The average pore size thereof was found to be 18.1 nm. X-ray diffraction (XRD) analysis showed that the catalyst 26 includes mixed powders of a rutile phase titania and a pseudobrookite (Fe₂TiO₅) phase titania and ceria.

Subsequently, chemical looping process according to one embodiment using the catalyst 26 was performed. Results of catalyst activity using the catalyst 26 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 27

### Production of Catalyst 27

(iron/cerium/titanium molar ratio=0.075/0.300/1, calcination temperature 700° C.)

A catalyst 27 was produced in the same manner as the Present Example 1 excerpt that 0.36 mmol of cerium chloride was used and the hot calcination condition was adjusted to 700° C. and the precipitate was maintained for 6 hours. The catalyst 27 was composed such that the iron/cerium/titanium molar ratio is 0.075/0.300/1. The specific surface area of the catalyst 27 was 6.8 m²/g. The average pore size thereof was found to be 17.2 nm. X-ray diffraction (XRD) analysis showed that the catalyst 27 includes mixed powders of a rutile phase titania and a pseudobrookite (Fe₂TiO₅) phase titania and ceria.

Subsequently, chemical looping process according to one embodiment using the catalyst 27 was performed. Results of catalyst activity using the catalyst 27 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 28

### Production of Catalyst 28

(iron/cerium/titanium molar ratio=0.075/0.300/1, calcination temperature 800° C.)

A catalyst 28 was produced in the same manner as the Present Example 1 excerpt that 0.36 mmol of cerium chloride was used and the hot calcination condition was adjusted to 800° C. and the precipitate was maintained for 6 hours. The catalyst 28 was composed such that the iron/cerium/titanium molar ratio is 0.075/0.300/1. The specific surface area of the catalyst 28 was 6.0 m²/g. The average pore size thereof was found to be 12.8 nm. X-ray diffraction (XRD) analysis showed that the catalyst 28 includes mixed powders of a rutile phase titania and a pseudobrookite (Fe₂TiO₅) phase titania and ceria.

Subsequently, chemical looping process according to one embodiment using the catalyst 28 was performed. Results of catalyst activity using the catalyst 28 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 29

### Production of Catalyst 29

(iron/cerium/titanium molar ratio=0.075/0.300/1, calcination temperature 900° C.)

A catalyst 29 was produced in the same manner as the Present Example 1 excerpt that 0.36 mmol of cerium chloride was used and the hot calcination condition was adjusted to 900° C. and the precipitate was maintained for 6 hours. The catalyst 29 was composed such that the iron/cerium/titanium molar ratio is 0.075/0.300/1. The specific surface area of the catalyst 29 was 3.6 m²/g. The average pore size thereof was found to be 9.4 nm. X-ray diffraction (XRD) analysis showed that the catalyst 29 includes mixed powders of a rutile phase titania and a pseudobrookite (Fe₂TiO₅) phase titania and ceria.

Subsequently, chemical looping process according to one embodiment using the catalyst 29 was performed. Results of catalyst activity using the catalyst 29 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### PRESENT EXAMPLE 30

### Production of Catalyst 30

(iron/cerium/titanium molar ratio=0.075/0.300/1, calcination temperature 1000° C.)

A catalyst 30 was produced in the same manner as the Present Example 1 excerpt that 0.36 mmol of cerium chloride was used and the hot calcination condition was adjusted to 1000° C. and the precipitate was maintained for 6 hours. The catalyst 30 was composed such that the iron/cerium/titanium molar ratio is 0.075/0.300/1. The specific surface area of the catalyst 30 was 1.3 m²/g. The average pore size thereof was found to be 10.5 nm. X-ray diffraction (XRD) analysis showed that the catalyst 30 includes mixed powders of a rutile phase titania and a pseudobrookite (Fe₂TiO₅) phase titania and ceria.

Subsequently, chemical looping process according to one embodiment using the catalyst 30 was performed. Results of catalyst activity using the catalyst 30 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### COMPARATIVE EXAMPLE 1

### Production of Catalyst 31

A reaction was carried out under the same reaction conditions as in Example 1. Commercially available titania having the anatase phase of the catalyst was used as a catalyst 31 (catalyst Comm-TiO₂ (Anatase)). The specific surface area of the catalyst 31 was 3.5 m²/g. An average pore size thereof was found to be 12.7 nm.

Subsequently, chemical looping process according to one embodiment using the catalyst 31 was performed. Results of catalyst activity using the catalyst 31 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### COMPARATIVE EXAMPLE 2

### Production of Catalyst 32

A reaction was carried out under the same reaction conditions as in Example 1. Commercially available titania having the rutile phase of the catalyst was used as a catalyst 32 (catalyst Comm-TiO₂ (Rutile)). The specific surface area of the catalyst 32 was 0.1 m²/g.

Subsequently, chemical looping process according to one embodiment using the catalyst 32 was performed. Results of catalyst activity using the catalyst 32 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### COMPARATIVE EXAMPLE 3

### Production of Catalyst 33

A reaction was carried out under the same reaction conditions as in Example 1. Commercial titanium iron (Ilmenite, FeTiO₃), and ceria (CeO₂) were physically mixed with each other to produce a catalyst 33 (Comm-FeTiO₃+CeO₂−1).

Subsequently, chemical looping process according to one embodiment using the catalyst 33 was performed. Results of catalyst activity using the catalyst 33 are shown in following tables based on amounts of products such as methane, ethylene, carbon dioxide, and carbon monoxide as generated by selective dehydrogenation of ethane and an amount of carbon monoxide (consumption of carbon dioxide) as produced by the conversion of carbon dioxide in the chemical looping process according to one embodiment.

### COMPARATIVE EXAMPLE 4

### Production of Catalyst 34

A catalyst 34 (Fe_{0.150}Ce_{0.075}TiOₓ) was produced in the same manner as the Present Example 1 excerpt that commercial titanium iron (Ilmenite, FeTiO₃), and ceria (CeO₂) were physically mixed with each other and 0.18 mmol of iron chloride was used, and the hot calcination condition was adjusted to 600° C. and the precipitate was maintained for 6 hours. The catalyst 34 was composed such that the iron/cerium/titanium molar ratio is 0.150/0.075/1. The specific surface area of the catalyst 34 was 34.9 m²/g. The average pore size thereof was found to be 9.1 nm. X-ray diffraction (XRD) analysis showed that the catalyst 34 includes powders of an anatase phase titania.

Subsequently, chemical looping process according to one embodiment using the catalyst 34 was performed. In this connection, the chemical looping process was performed under the same reaction conditions as the Present Example 1, but the reaction was conducted at 800° C. using methane gas as a reducing reaction gas. The results for the catalyst activity for the combustion reaction of methane using the catalyst 34 are shown in the following tables.

### COMPARATIVE EXAMPLE 5

### Production of Catalyst 35

Commercial titanium iron (Ilmenite, FeTiO₃), and ceria (CeO₂) were physically mixed with each other to produce a catalyst 35 (Comm-FeTiO₃+CeO₂−2). Subsequently, chemical looping process according to one embodiment using the catalyst 35 was performed. In this connection, the chemical looping process was performed under the same reaction conditions as the Present Example 1, but the reaction was conducted at 800° C. using methane gas as a reducing reaction gas. The results for the catalyst activity for the combustion reaction of methane using the catalyst 35 are shown in the following tables.

### Reaction Example

### Chemical Looping Process

Subsequently, chemical looping process according to one embodiment using each of the catalysts 1 to 35 was performed. In order to perform the reaction, an Inconel fixed bed reactor having an inner diameter of 7 mm, an outer diameter of 9.5 mm, and a height of 420 mm were used. A temperature inside the reactor was measured after placing a 1/16 inch thermocouple from a top of the reactor into the reactor and positioning the thermocouple above the catalyst bed. A 207 mm long 4/16 tube was disposed from a bottom of the reactor in order to place the catalyst in the center of the reactor. Then, 1 g of each catalyst, and 0.15 g of glass wool were charged into the reactor through the tube. Then, the reaction according to one embodiment was carried out. In this connection. The reactor was supplied with heat using an electrical furnace (World Energy company). The thermocouple external to the furnace was used to measure the temperature. Then, the temperature of the reactor may be externally controlled by a PID controller based on the measured temperature.

Only nitrogen was injected to the reactor while increasing the reactor temperature from room temperature to a reduction temperature of 550° C. Then, an isothermal condition is established in which the reduction temperature in accordance with the present disclosure is maintained at 550° C. Then, a total gas flow rate into the reactor was changed to a 30 cc/min to adjust a content of the ethane gas to 20 mol % (ethane 6 cc/min, nitrogen 24 cc/min), and then, a reduction reaction was performed for 4 hours.

After the reduction reaction was completed, the flow of ethane was shut off and only nitrogen was flowed into the reactor to remove residual gas inside the reactor. Thereafter, the temperature inside the reactor was set to 700° C. Then, the oxidation reaction was performed under a nitrogen gas atmosphere having the same gas flow rate as in the reduction reaction and containing 20 mol % of carbon dioxide.

Termination of the oxidation reaction was calculated based on a timing at which generation of carbon monoxide as a product was not identified by GC (gas chromatography) as an analyzer. The produced gas was analyzed by gas chromatography connected to the reactor. A thermal conductivity detector (TCD) and flame ionization detector (FID) were used.

TABLE 1

| | calcination | specific surface | |
|---|---|---|---|
| | temperature | area (m²/g)/ | |
| Examples | (° C. ) | pore size (nm) | Catalyst |
| Present | 600 | 48.5/9.4 | Fe_{0.075}Ce_{0.075}TiOₓ |
| Example 1 | | | |
| Present | 700 | 23.6/12.4 | |
| Example 2 | | | |
| Present | 800 | 0.69/13.5 | |
| Example 3 | | | |
| Present | 900 | 0.23/37.9 | |
| Example 4 | | | |
| Present | 1000 | 0.07/8.5 | |
| Example 5 | | | |
| Present | 600 | 34.9/9.1 | Fe_{0.150}Ce_{0.075}TiOₓ |
| Example 6 | | | |
| Present | 700 | 7.5/11.1 | |
| Example 7 | | | |
| Present | 800 | 0.17/39.8 | |
| Example | 8 | | |
| Present | 900 | 0.03/10.4 | |
| Example 9 | | | |
| Present | 1000 | 0.02/— | |
| Example 10 | | | |
| Present | 600 | 44.3/16.3 | Fe_{0.225}Ce_{0.075}TiOₓ |
| Example 11 | | | |
| Present | 700 | 17.5/17.9 | |
| Example 12 | | | |
| Present | 800 | 6.03/12.7 | |
| Example 13 | | | |
| Present | 900 | 3.26/23.8 | |
| Example 14 | | | |
| Present | 1000 | 1.18/33.0 | |
| Example 15 | | | |
| Present | 600 | 50.6/13.1 | Fe_{0.300}Ce_{0.075}TiOₓ |
| Example 16 | | | |
| Present | 700 | 18.3/20.2 | |
| Example 17 | | | |
| Present | 800 | 3.81/20.4 | |
| Example 18 | | | |
| Present | 900 | 2.25/8.6 | |
| Example 19 | | | |
| Present | 1000 | 0.82/8.6 | |
| Example 20 | | | |
| Present | 600 | 31.0/12.5 | Fe_{0.075}Ce_{0.150}TiOₓ |
| Example 21 | | | |
| Present | 700 | 19.9/17.7 | |
| Example 22 | | | |
| Present | 800 | 7.2/22.3 | |
| Example 23 | | | |
| Present | 900 | 2.8/34.9 | |
| Example 24 | | | |
| Present | 1000 | 1.0/33.3 | |
| Example 25 | | | |
| Present | 600 | 9.3/18.1 | Fe_{0.075}Ce_{0.300}TiOₓ |
| Example 26 | | | |
| Present | 700 | 6.8/17.2 | |
| Example 27 | | | |
| Present | 800 | 6.0/12.8 | |
| Example 28 | | | |
| Present | 900 | 3.6/9.4 | |
| Example 29 | | | |
| Present | 1000 | 1.3/10.5 | |
| Example 30 | | | |
| Comparative | — | 3.5/12.7 | Comm-TiO₂ (anatase) |
| Example 1 | | | |
| Comparative | — | 0.1/— | Comm-TiO₂ (rutile) |
| Example 2 | | | |
| Comparative | — | —/— | Comm-FeTiO₃ + CeO₂ |
| Example 3 | | | |
| Comparative | 600 | 34.9/9.1 | Fe_{0.150}Ce_{0.075}TiOₓ |
| Example 4 | | | |
| Comparative | — | —/— | Comm-FeTiO₃ + CeO₂ |
| Example 5 | | | |

TABLE 2

| | | | | | | | | CO₂ active |
|---|---|---|---|---|---|---|---|---|
| | | | | | | | | reaction |
| | C₂H₆ dehydrogenation reaction | | | | | | | CO |
| | C₂H₆ | Selectivity (carbon mol %) | | | | | Yield | generation |
| Examples | conversion(%) | CO | CO₂ | CH₄ | C₂H₄ | C3-C4 | (%) | (mmol/g_{cat}) |
| Present Example 1 | 6.1 | 5.6 | 0.0 | 3.9 | 90.4 | 0.1 | 5.7 | 0.08 |
| Present Example 2 | 6.4 | 0.0 | 6.1 | 6.0 | 87.7 | 0.0 | 5.8 | 0.04 |
| Present Example 3 | 4.4 | 0.0 | 11.0 | 4.4 | 84.6 | 0.0 | 4.3 | 0.04 |
| Present Example 4 | 2.3 | 0.0 | 18.0 | 3.4 | 78.6 | 0.0 | 2.2 | 0.00 |
| Present Example 5 | 0.8 | 0.0 | 27.9 | 2.3 | 69.8 | 0.0 | 1.2 | 0.00 |
| Present Example 6 | 17.6 | 7.8 | 8.8 | 12.4 | 70.8 | 0.9 | 8.3 | 0.49 |
| Present Example 7 | 3.0 | 2.1 | 0.7 | 3.9 | 93.3 | 0.0 | 0.6 | 0.08 |
| Present Example 8 | 0.7 | 1.2 | 3.8 | 1.0 | 94.0 | 0.0 | 0.5 | 0.01 |
| Present Example 9 | 1.3 | 0.0 | 2.9 | 1.5 | 95.7 | 0.0 | 1.3 | 0.03 |
| Present Example 10 | 1.3 | 0.0 | 0.0 | 1.7 | 98.3 | 0.0 | 1.3 | 0.28 |
| Present Example 11 | 14.0 | 6.0 | 8.5 | 9.4 | 75.4 | 0.0 | 6.2 | 0.39 |
| Present Example 12 | 7.8 | 5.5 | 4.1 | 5.3 | 84.9 | 0.0 | 6.2 | 0.08 |
| Present Example 13 | 4.2 | 5.3 | 4.3 | 4.1 | 86.3 | 0.0 | 3.5 | 0.00 |
| Present Example 14 | 7.7 | 8.8 | 6.8 | 3.5 | 80.9 | 0.0 | 4.7 | 0.02 |
| Present Example 15 | 1.3 | 15.3 | 4.0 | 3.4 | 77.3 | 0.0 | 1.0 | 0.04 |
| Present Example 16 | 6.2 | 11.8 | 3.2 | 9.2 | 75.6 | 0.4 | 4.5 | 0.00 |
| Present Example 17 | 4.3 | 10.3 | 5.4 | 5.4 | 78.9 | 0.0 | 3.4 | 0.02 |
| Present Example 18 | 3.9 | 5.1 | 10.1 | 6.4 | 77.9 | 0.0 | 3.1 | 0.03 |
| Present Example 19 | 1.1 | 0.0 | 18.5 | 3.1 | 78.4 | 0.0 | 0.9 | 0.00 |
| Present Example 20 | 2.3 | 10.8 | 19.7 | 4.7 | 64.9 | 0.0 | 1.4 | 0.00 |
| Present Example 21 | 10.8 | 14.1 | 9.7 | 4.2 | 72.0 | 0.9 | 3.1 | 0.26 |
| Present Example 22 | 6.6 | 14.9 | 4.9 | 4.3 | 75.0 | 0.0 | 4.0 | 0.43 |
| Present Example 23 | 4.8 | 4.9 | 6.5 | 0.4 | 88.2 | 0.0 | 4.0 | 0.00 |
| Present Example 24 | 2.2 | 1.0 | 3.8 | 8.7 | 86.4 | 0.0 | 2.0 | 0.00 |
| Present Example 25 | 0.9 | 0.0 | 3.4 | 2.3 | 94.3 | 0.0 | 0.8 | 0.00 |
| Present Example 26 | 6.3 | 15.0 | 14.2 | 4.2 | 66.7 | 2.2 | 3.9 | 0.27 |
| Present Example 27 | 4.3 | 17.1 | 6.0 | 4.5 | 72.5 | 1.2 | 2.7 | 0.01 |
| Present Example 28 | 2.1 | 20.2 | 27.7 | 0.1 | 52.0 | 0.0 | 1.1 | 0.01 |
| Present Example 29 | 2.0 | 19.6 | 14.1 | 4.3 | 62.0 | 0.0 | 1.0 | 0.05 |
| Present Example 30 | 0.9 | 1.2 | 26.4 | 0.1 | 72.3 | 0.0 | 0.9 | 0.00 |
| Comparative Example 1 | 0.5 | 0 | 0 | 0 | 100 | 0.0 | 0.5 | 0.00 |
| Comparative Example 2 | 0.1 | 0 | 0 | 0 | 100 | 0.0 | 0.1 | 0.02 |
| Comparative Example 3 | 1.3 | 33.4 | 3.0 | 5.5 | 58.1 | 0.0 | 0.8 | 0.15 |

| | |
|---|---|
| | CH₄ combustion reaction |
| | CH₄ conversion (%) |
| Comparative Example 4 | 62.2 |
| Comparative Example 5 | 36.9 |

Referring to Table 1, and Table 2, the catalysts produced according to Present Examples 1 to 30, as shown above, refer to a heterogeneous catalyst proposed by the present disclosure and are composed such that each of the molar ratios of iron and cerium to titanium is in a range of 0.075 to 0.300.

When analyzing the catalysts produced according to embodiments with each other, the specific surface areas thereof are in the range of about 20 to 50 m²/g, while the pore size of the catalysts is in the range of 6 to 18 nm. When the calcination temperature is in a range of 600 to 700° C., the consumption of ethane and carbon dioxide is particularly predominant. Thus, the conversion of ethane over the entire reduction reaction time is 6% or greater. In this connection, the selectivity of ethylene is 70% or greater and the yield was at least 4% and a total consumption of carbon dioxide is 0.04 mmol/g or greater. Further, in the conventional chemical looping process using the methane, the catalyst of Comparative Example 4 as produced according to one embodiment of the present disclosure has a higher methane conversion of 62% compared to the conventional commercial catalyst of Comparative Example 5.

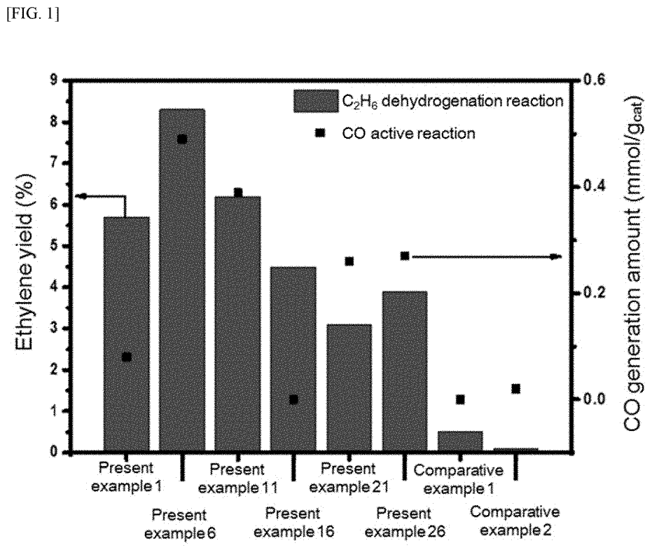

Further, FIG. 1 is a diagram showing the results of the chemical looping process according to one embodiment of the present disclosure. Specifically, FIG. is a graph showing the ethylene yields of the catalysts of Present Example 6, Present Example 11, Present Example 1, Present Example 16, Present Example 26, and Present Example 21. In case of Present Example 6, and Present Example 11, the dehydrogenation reaction of ethane is active, such that high ethylene yield is achieved, and carbon dioxide activity is high, such that carbon monoxide production was found to be quite high. On the other hand, in the case of the commercial titania based catalysts in which the iron oxide is not contained (that is, in Comparative Example 1, and Comparative Example 2), dehydrogenation reaction level of ethane is low and carbon dioxide activity is low. In particular, it was confirmed that the ethane conversion is lower than 0.3%.

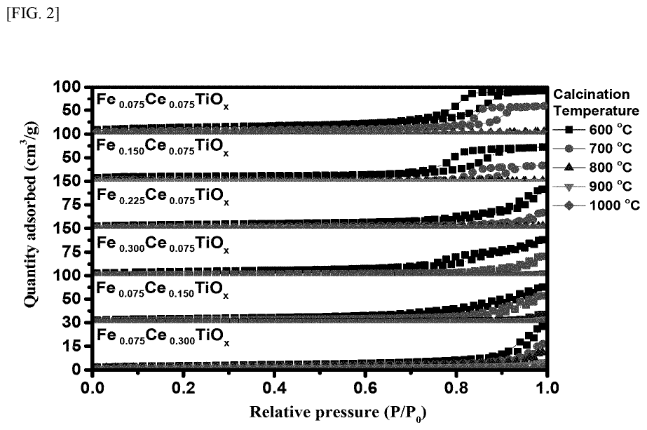

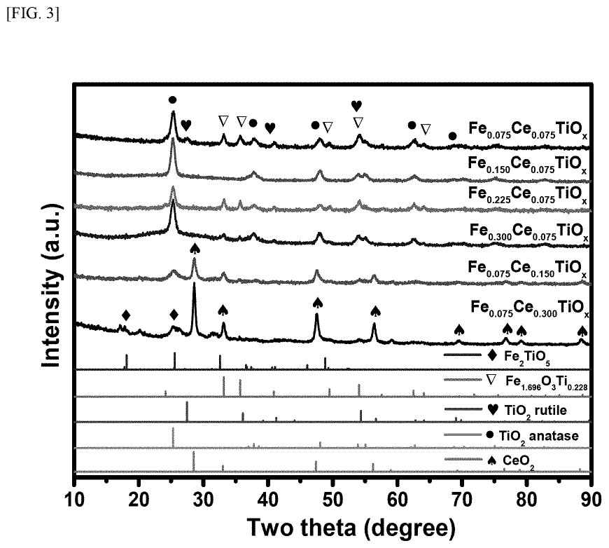

FIG. 2 and FIG. 3 shows the results of catalyst analysis according to one embodiment of the present disclosure. Specifically, FIG. 2 shows the nitrogen adsorption and desorption isotherm of the catalysts according to one embodiment of the present disclosure. That is, FIG. 2 shows nitrogen adsorption, and desorption degree, based on a relative pressure, of the catalysts produced based on changes in the calcination temperature, and metal content. While the relative pressure (P/P₀) varies to between 0 to 1 bar at a liquid nitrogen temperature (77K, −196 degrees C.), the amount of physically adsorbed nitrogen gas onto the surface and into the pores of the catalyst is measured. Thus, it was confirmed that the specific surface areas of the catalysts were in the range of 20 m²/g to 50 m²/g, while the pore sizes of the catalysts were in a range of 6 to 18 nm. When the molar ratio of iron to titanium is in a range of 0075 to 0.150, and the calcination temperature is in a range of 600° C. to 700° C., the catalyst has a type IV adsorption isotherm. As the calcination temperature is high, the specific surface area of the catalyst is smaller.

The XRD analysis using X'Pert PRO apparatus determines the crystal structure of the catalyst corresponding to the calcination temperature of 600° C. The result thereof is shown in FIG. 3. A wavelength of CuKα (1.54 Å) was used. Used voltage and current were specifically 30 mA and 40 kV. The crystal structure was measured in a range of 2θ=5-90 degrees at a scanning rate of 7.72 degrees/min. The catalyst produced when the molar ratio of iron to titanium is in a range of 0.075 to 0.300 includes mixed powders of titania, and Fe_{1.696}O₃Ti_{0.228}. The catalyst produced when a molar ratio of cerium to titanium is in a range of 0.150 to 0.300 includes mixed powders of ceria and Fe₂TiO₅ phase.

When using the catalyst in accordance with the present disclosure, CO₂/NO_{X} emissions, and energy may be reduced and the yield of ethylene may be improved via producing the water using the selective oxidation of hydrogen. In particular, since the catalyst in accordance with the present disclosure includes the complex metal oxide containing the oxygen donor particles, a separate oxygen supply, and a separate oxygen separation unit (ASU) maybe not required. Accordingly, the process equipment cost, and operating cost may be reduced. The flammable oxygen may be separated from the gas used as the fuel.

In accordance with the present disclosure, the chemical looping process technology may be used to independently perform olefin generation via selective dehydrogenation of paraffin and carbon monoxide generation via reduction of carbon dioxide. Further, in accordance with the present disclosure, a disadvantage of the conventional general chemical looping process, and partial oxidation reaction that it was difficult to control the generation of carbon dioxide may be removed to increase the yield of olefins via the conversion of paraffin. Further, the conversion of carbon dioxide (Carbon dioxide, CO2) as a major source of greenhouse gases may produce carbon monoxide as a reaction intermediate that can synthesize useful chemicals.

Although the present disclosure has been described with reference to the preferred embodiments of the present disclosure, those skilled in the art may understand that the present disclosure may be variously modified and changed without departing from the spirit and scope of the present disclosure as described in the claims below.

What is claimed is:

1. A catalyst for chemical looping process, wherein the chemical looping produces ethylene, and carbon monoxide from ethane, and carbon dioxide, wherein the catalyst comprise a complex metal oxide containing iron (Fe), cerium (Ce), and titanium (Ti).

2. The catalyst for chemical looping process of claim 1, wherein the complex metal oxide is composed such that a molar ratio of iron (Fe) to titanium (Ti) is in a range of 0.075 to 0.3, and a molar ratio of cerium (Ce) to titanium (Ti) is in a range of 0.075 to 0.3.

3. The catalyst for chemical looping process of claim 2, wherein the complex metal oxide contains at least one of a rutile phase titania or an anatase phase titania.

4. The catalyst for chemical looping process of claim 3, wherein the complex metal oxide contains ceria.

5. The catalyst for chemical looping process of claim 4, wherein the complex metal oxide contains FeTi oxide containing iron, and titanium.

6. The catalyst for chemical looping process of claim 1, wherein the complex metal oxide is composed of a carrier, and an active component substituted onto the carrier, wherein the carrier includes titania.

7. The catalyst for chemical looping process of claim 6, wherein the active component includes a perovskite-structured material made of iron and cerium substituted onto titanium.

8. The catalyst for chemical looping process of claim 6, wherein the catalyst has a conversion of ethane of 0.7% or greater, and has an ethylene selectivity of 62.0% or greater.

9. A method for producing a catalyst for chemical looping process, the method comprising:
producing a first mixed solution by mixing iron precursor and cerium precursor with an aqueous solution containing titanium precursor;
producing a second mixed solution by adding urea to the first mixed solution;
producing first precipitate by cooling and filtering the second mixed solution; and
producing a complex metal oxide by performing hot calcination of the first precipitate.

10. The method of claim 9, wherein the titanium precursor includes titanium oxysulfate (TiOSO₄).

11. The method of claim 10, wherein the iron precursor includes iron chloride (FeCl₃).

12. The method of claim 11, wherein the cerium precursor includes chloride cerium (CeCl₃).

13. The method of claim 12, wherein the hot calcination is carried out at a temperature range of 500° C. to 1100° C.

14. A method for producing ethylene and carbon monoxide, the method comprising:
disposing of the catalyst for chemical looping process according to claim 1 inside a chemical looping reactor;
first heating the reactor to a reduction temperature or higher;
injecting ethane-containing gas into the reactor to perform a reduction reaction;
second heating the reactor; and
injecting carbon dioxide-containing gas into the reactor to perform an oxidation reaction.

15. The method of claim 14, wherein the first heating is carried out to a range of 450° C. to 650° C.

16. The method of claim 14, further comprising:
after the injecting of the ethane-containing gas and before the second heating, converting an inside of the reactor into an inert gas atmosphere.

17. The method of claim 14, wherein the second heating is carried out to a range of 600° C. to 800° C.

18. The method of claim 14, wherein the disposing of the catalyst, the first heating, the injecting of the ethane-containing gas, the second heating and the injecting of the carbon dioxide-containing gas are sequentially repeated.
